(19) United States
(12) Patent Application Publication
(10) Pub. No.: US 2018/0161014 A1
(43) Pub. Date: Jun. 14, 2018

(54) Ultrasonic CT Apparatus

(71) Applicant: Hitachi, Ltd., Tokyo (JP)

(72) Inventors: Wenjing WU, Tokyo (JP); Kenichi KAWABATA, Tokyo (JP); Yushi TSUBOTA, Tokyo (JP); Takahide TERADA, Tokyo (JP); Atsurou SUZUKI, Tokyo (JP); Kazuhiro YAMANAKA, Tokyo (JP)

(21) Appl. No.: 15/832,232

(22) Filed: Dec. 5, 2017

(30) Foreign Application Priority Data
Dec. 12, 2016 (JP) 2016-240576

(51) Int. Cl. A61B 8/08; A61B 8/00; A61B 8/15

(52) U.S. Cl. CPC A61B 8/5261; A61B 8/54; A61B 8/4477; A61B 6/0435; A61B 8/406; A61B 8/15; A61B 8/0825

(57) ABSTRACT

An error in a physical property value due to refraction of an ultrasonic wave is reduced, and thus the physical property value is calculated with high accuracy. A transducer of a transducer array transmits an ultrasonic beam to a subject placed in a predetermined imaging space, the transducer receives a transmission of the ultrasonic beam transmitted through the subject, and an image generating unit generates a transmission image based on the reception signal. The controller includes a focal length adjusting unit that adjusts a length between a focal position of the ultrasonic beam and the center of an imaging space to adjust a divergence angle of the ultrasonic beam.

### TECHNICAL FIELD

The present invention relates to an ultrasonic imaging apparatus, particularly, to an ultrasonic CT apparatus that generates an image of a subject using an ultrasonic wave transmitted through the subject.

### BACKGROUND ART

There has been known an ultrasonic imaging apparatus in which a subject placed in a medium is irradiated with ultrasonic waves in a plurality of directions, a physical property value (a sound speed or an attenuation rate) in the subject is obtained from a transmission signal transmitted through the subject, and a tomographic image is generated with the physical property value. This apparatus is referred to as an ultrasonic computed tomography (CT) apparatus. PTL 1 discloses a basic configuration and an imaging technique of an ultrasonic CT apparatus. In the apparatus in PTL 1, a subject is inserted into an opening around which acoustic transducers are arranged in a ring array shape, one transducer generates an ultrasonic wave with which the subject is irradiated, and another ultrasonic element receives a transmission. The operation is repeatedly performed while the transducers that transmit ultrasonic waves are shifted one by one, and an obtained reception signal is reconstructed. In this manner, a tomographic image of a sound speed or an attenuation rate is generated.

### CITATION LIST

### Patent Literature

PTL 1: JP-A-8-508925

### SUMMARY OF INVENTION

### Technical Problem

As PTL 1, in a case where an ultrasonic wave is transmitted from one transducer, the transmitted ultrasonic wave forms a fan beam that is diverged at a predetermined angle to form a fan shape, and a subject is irradiated therewith. Therefore, transmissions reach and are received in a ring array having a range which is wider than a size of the subject, and thus it is possible to obtain an effect of magnified projection and to increase resolution. However, the upper limit of the intensity of an ultrasonic wave that can be transmitted from one transducer is determined depending on a structure of the transducer, a withstand voltage of a signal line through which a transmission voltage is supplied to the transducer, or the like, and thus it is not possible to transmit a fan beam having intensity equal to or higher than the upper limit. Since the intensity of a reception signal, which is received by one transducer that receives the ultrasonic waves, depends on the intensity of the transmitted ultrasonic wave, it is difficult to increase the intensity of the reception signal, and it is difficult to increase accuracy of a reconstructed image.

An object of the present invention is that the intensity of an ultrasonic wave, which is transmitted, is increased while an effect of magnified projection is obtained with a fan beam, and thus accuracy of the ultrasonic image is increased.

### Solution to Problem

In order to achieve the object described above, an ultrasonic CT apparatus of the present invention includes: a transducer array having an arrangement of a plurality of transducers that transmit an ultrasonic beam to a subject placed in a predetermined imaging space, receive a transmission of the ultrasonic beam transmitted through the subject, and output a reception signal; an image generating unit that generates a transmission image based on the reception signal of the transducer array; and a controller that controls the ultrasonic beam that is transmitted from the transducer array. The controller includes a focal length adjusting unit that adjusts a length between a focal position of the ultrasonic beam and the center of an imaging space to adjust a divergence angle of the transmitted ultrasonic beam.

### Advantageous Effects of Invention

According to the present invention, the intensity of an ultrasonic wave, which is transmitted, is increased while an effect of magnified projection is obtained, and thus accuracy of the ultrasonic image is increased.

### BRIEF DESCRIPTION OF DRAWINGS

FIG. 1 is a block diagram schematically illustrating the entire configuration of an ultrasonic imaging apparatus of an embodiment of the present invention.

FIG. 2 is a view illustrating a state of a transducer array including a plurality of transducers of the embodiment when viewed from below.

FIGS. 3A and 3B are views illustrating a fan beam for irradiation and a focal position thereof according to the embodiment, FIG. 3C is a view illustrating a fan beam in the related art, and FIG. 3D is a view illustrating a parallel beam.

FIG. 4 is a diagram illustrating slices set in a subject.

FIG. 5 is a functional block diagram illustrating a function of an image generating unit of Embodiment 1.

FIG. 6 is a sequence diagram illustrating transmission and reception of a signal of units of an ultrasonic CT apparatus of Embodiment 1.

FIG. 7 is an example of a screen for receiving an operation by a user in the apparatus of Embodiment 1.

FIG. 8 is a flowchart illustrating an operation of a controller of Embodiment 1.

FIG. 9 is a focal length reference table that is stored in a storage unit of Embodiment 1.

FIG. 10 is a transmission/reception setting condition table that is stored in the storage unit of Embodiment 1.

FIGS. 11A and 11B illustrate examples of images with a defect, and FIG. 11C illustrates an example of an image obtained by removing a defect.

FIGS. 12A and 12B are views illustrating a shape of a fan beam during imaging of a transmission image of Embodiment 5.

FIG. 13 is a view illustrating irradiation with fan beams having different focal lengths alternately for each view for the same slice according to Embodiment 5.

FIG. 14 is a view illustrating imaging of a flying focal spot of Embodiment 6.

FIG. 15 is a view illustrating a fan beam having a focal position between arranged transducers of Embodiment 7.

FIG. 16A is a view illustrating a fan beam of which a focal position is adjusted depending on an FOV that is large to the extent that the entirety of a subject is included according to Embodiment 8, FIG. 16B illustrates an example of a transmission image of the entirety of the subject, FIG. 16C is a view illustrating a fan beam of which a focal position is adjusted depending on the FOV that is small to the extent that only an ROI of a subject is included, and FIG. 16D illustrates an example of a transmission image of the ROI.

FIG. 17 is a sequence diagram illustrating transmission and reception of a signal of units of Embodiment 8.

FIG. 18 is a flowchart illustrating operations of units of Embodiment 8.

### DESCRIPTION OF EMBODIMENTS

Hereinafter, an embodiment of the present invention will be described with reference to the figures.

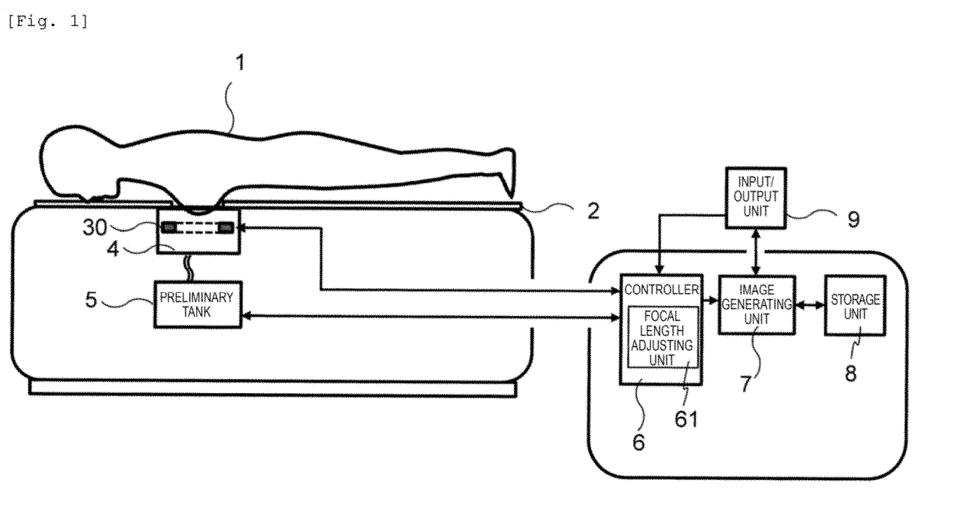

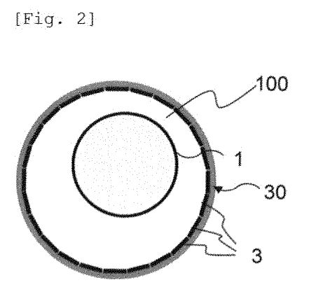

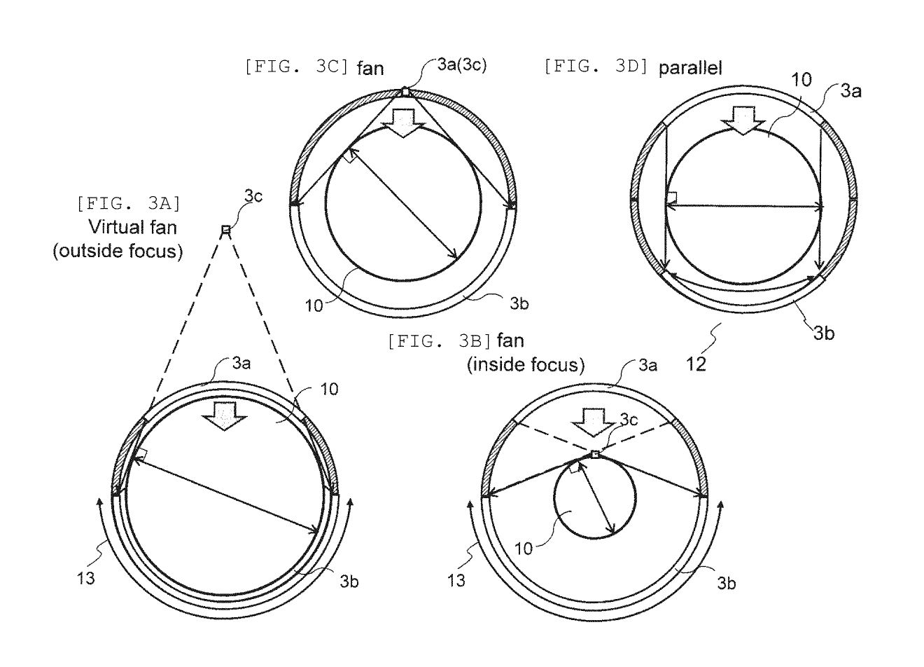

FIG. 1 schematically illustrates the entire configuration of an ultrasonic CT apparatus (ultrasonic imaging apparatus) of the embodiment. FIG. 2 is a view illustrating a state of a transducer array 30 including a plurality of transducers 3 when viewed from below. FIGS. 3 (a) and 3 (b) are views illustrating a fan beam for irradiation and a focal position thereof according to the embodiment. In FIG. 1, a case where an imaging site of a subject 1 is the breast is illustrated as an example; however, an imaging target of the ultrasonic CT apparatus of the embodiment is not limited to the breast. In addition, a focus may be referred to as a sound source.

As illustrated in FIG. 1, the ultrasonic CT apparatus of the embodiment includes the transducer array 30, a controller 6, and an image generating unit 7. As illustrated in FIG. 2, the transducer array 30 has a structure in which a plurality of transducers are arranged, transmit an ultrasonic beam to the subject 1 placed in a predetermined imaging space 100, receive a transmission of the ultrasonic beam transmitted through the subject 1, and output a reception signal. The controller 6 controls the ultrasonic beam that is transmitted from the transducer array 30. The image generating unit 7 generates a transmission image based on the reception signal of the transducer array 30.

Here, the controller 6 includes a focal length adjusting unit 61. As illustrated in FIGS. 3(a) and 3(b), the focal length adjusting unit 61 adjusts a length between a focal position 3c of the ultrasonic beam and the center of the imaging space 100, thereby adjusting a divergence angle of the ultrasonic beam. For example, the focal length adjusting unit 61 adjusts the focal length, thereby setting the ultrasonic beam to a fan beam.

As described above, the ultrasonic beam, of which the focal position is adjusted, is transmitted, and thereby the fan beam is formed from the ultrasonic wave transmitted from the plurality of transducers 3a as illustrated in FIGS. 3(a) and 3(b). Accordingly, compared to a case where the fan beam is transmitted from a single transducer 3a as illustrated in FIG. 3(c), in the ultrasonic CT apparatus of the embodiment, it is possible to transmit a fan beam having high intensity. Hence, in the ultrasonic CT apparatus of the embodiment, it is possible to increase the intensity of the ultrasonic wave, which is transmitted through the subject 1 placed in the FOV (imaging field of view) 10 in an irradiation region of fan beam and is received by a transducer 3b, and it is possible to improve accuracy of the transmission image. Moreover, as illustrated in FIGS. 3(a) and 3(b), in a case where the fan beam is transmitted to the subject 1, a rate of a range 13 of the transducer 3b that the transmission reaches to a size of the subject 1 (magnification ratio by projection) is larger than 1 and is larger than a magnification ratio (=1) through projection in a case where a parallel beam is transmitted as illustrated in FIG. 3(d). Accordingly, in a case where the subject 1 having the same size is imaged with the fan beam, the number of transducers 3b, which receive the transmissions, is larger than the number of parallel beams. Therefore, it is possible to improve the resolution of the transmission image. In other words, the ultrasonic CT apparatus of the embodiment can have both of an effect of increasing the intensity of the ultrasonic beam that is transmitted and an effect of improving the resolution by the magnified projection. In the embodiment, the ultrasonic beam, which is transmitted, is not limited to the fan beam. The parallel beam may be transmitted by setting the focal position to an infinite point.

The focal length adjusting unit 61 may virtually set the focal position 3c of the ultrasonic beam to a position which is farther away than the transducer array 30 (FIG. 3(a)) or a position in the imaging space 100 (FIG. 3(b)) when viewed from the center 100a of the imaging space 100. In this case, the controller 6 may perform control such that the ultrasonic waves that are output from the plurality of transducers 3a are superimposed on each other and the fan beam is formed. The focal length adjusting unit 61 adjusts a timing when the plurality of transducers 3a transmit ultrasonic waves, that is, adjusts a length between the focal position 3c of the ultrasonic beam and the center 100a of the imaging space 100 by adjusting an amount of delay. When the image generating unit 7 performs computation of image generation based on the divergence angle of the ultrasonic beam adjusted by the focal length adjusting unit 61 and reconstructs the transmission image in response to the reception signal obtained, based on the fan beam of which the divergence angle is adjusted, when the transmission image is generated.

As described above, in the embodiment, the length between a focal position 3c of the ultrasonic beam and the center 100a of the imaging space 100 is adjusted, and thereby a size of the FOV 10 is adjusted in the embodiment. In particular, in a case where the number of receiving transducers is constant, and the focal position 3c of the ultrasonic beam is virtually set to a position (FIG. 3(a)) which is farther away than the transducer array 30, a larger FOV 10 is obtained than that in a case where the ultrasonic wave is transmitted from the single transducer 3a as illustrated in FIG. 3(c), when viewed from the center 100a of the imaging space 100.

The focal length adjusting unit 61 changes the length between the focal position 3c and the center 100c of the imaging space 100 depending on the size of the subject 1 such that the entirety of the subject 1 is within the divergence angle of the fan beam. In this manner, whatever size the subject 1 has, it is possible to perform the irradiation with almost all of the ultrasonic waves output from the plurality of transducers 3a, and thus it is possible to increase the intensity of ultrasonic waves with which the subject 1 is irradiated. Accordingly, it is possible to improve the accuracy of the transmission image generated by the image generating unit.

The size of the subject 1 may be obtained by the focal length adjusting unit 61 based on an image generated by the image generating unit 7 and an image of the subject 1 which is imaged by another device. For example, the apparatus may have a configuration in which the transducer array further receives a reflected wave of the ultrasonic beam reflected from the subject 1, the image generating unit 7 generates a reflection image based on a reception signal of the reflected wave, and the focal length adjusting unit 61 obtains the size of the subject 1 based on the reflection image.

The apparatus may have a configuration in which the size of the subject 1 is received by a user. For example, as illustrated in FIG. 1, the ultrasonic CT apparatus is configured to further include an input unit (input/output unit 9), and the focal length adjusting unit 61 changes the length between the focal position 3c and the center 100a of the imaging space 100 depending on the size of the subject 1 which is received via the input/output unit 9.

FIG. 4 is a view illustrating slices in a case where the image generating unit 7 generates the transmission image for each of a plurality of slices 41 set for the subject 1. In a case where the image generating unit 7 is configured to generate the transmission image for each slice 41 of the subject 1, the focal length adjusting unit 61 may change the length between the focal position 3c and the center 100a of the imaging space 100 for each slice. In this manner, the FOV is set depending on the size of the subject 1 for each slice.

The controller 6 may change the length between the focal position 3c and the center 100a of the imaging space 100, thereby changing the FOV into FOVs having different sizes while the same number of transducers 3a which transmit the ultrasonic waves is maintained. Otherwise, both of the number of transducers 3a which transmit the ultrasonic waves and the length between the focal position 3c and the center 100a of the imaging space 100 are changed, and thereby the amount of change in the length between the focal position 3c and the center 100a of the imaging space 100 may be reduced while a range in which it is possible to change the FOV is increased.

### Embodiment 1

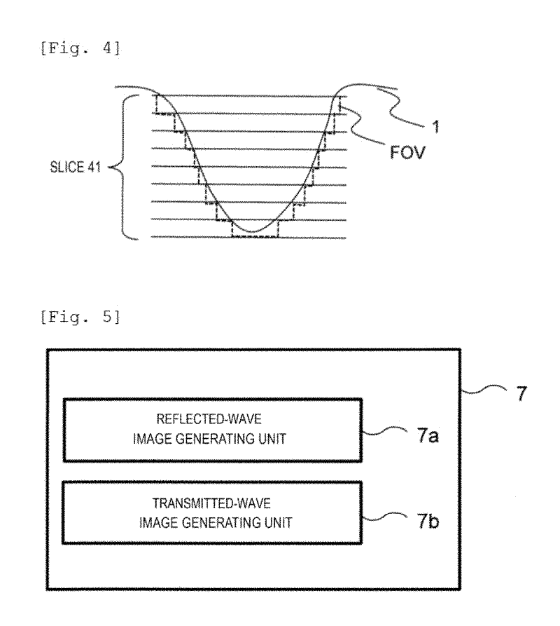

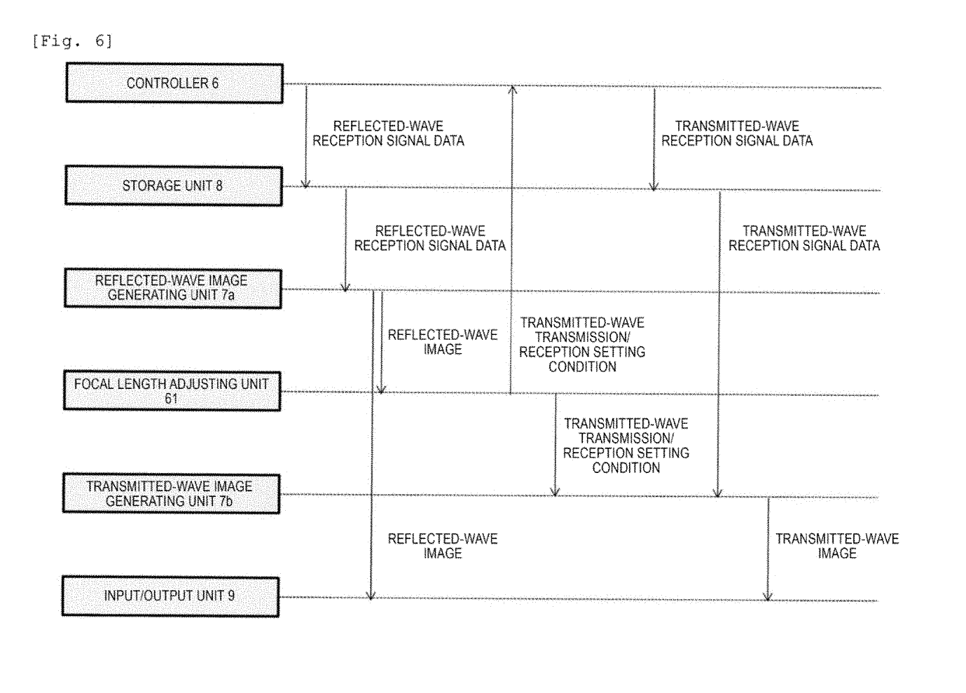

Hereinafter, an ultrasonic CT apparatus of a specific embodiment of the embodiment will be described. Here, an apparatus that acquires a transmission image of the breast is described as an example of the ultrasonic CT apparatus. FIG. 5 is a functional block diagram illustrating a function of the image generating unit 7.

As illustrated in FIG. 1, the ultrasonic CT apparatus of the embodiment includes a bed 2 on which the subject 1 is placed, a water tub 4, and a storage unit 8, in addition to the transducer array 30, the controller 6, the image generating unit 7, and the input/output unit 9 which are already described. As illustrated in FIG. 5, the image generating unit 7 includes a reflection image generating unit 7a and a transmission image generating unit 7b. The bed 2 has an opening in a surface on which the subject 1 is mounted and includes, below the opening, the cylindrical water tub 4 into which it is possible to insert the chest. In the inside thereof, the ring-shaped transducer array 30 as illustrated in FIG. 2 is provided to be movable in parallel in an axial direction of the water tub 4. The transducer array 30 has a configuration in which the transducers (piezoelectric elements or the like) 3 which are ultrasonic transceivers are arranged in a ring shape. The water tub 4 is filled with hot water and is connected to a preliminary tank 5. The preliminary tank 5 purifies, heats, and deaerates the hot water in the water tub 4. A thermometer (not illustrated) is attached to the inside of the preliminary tank 5 or a lower portion of the water tub 4 and is connected to the controller 6.

Imaging conditions of the ultrasonic CT apparatus are set by a user through a touch panel, a keyboard, or the like of the input/output unit 9. The set conditions or the like are stored in a memory or a hard disk drive which is the storage unit 8.

The controller 6 generates a transmission signal and outputs the transmission signal to transducers 3 constituting the transducer array 30, based on a condition input from the input/output unit 9 or an imaging condition stored in the storage unit 8. In this manner, the transducer 3 that receives the transmission signal outputs respective ultrasonic waves, thereby forming fan beams and transmitting the beam toward the subject 1. The transducer 3 that receives the ultrasonic waves transmitted through the subject 1 outputs the reception signal and the reception signal is input to the controller 6. The controller 6 includes a switching unit that switches between transmission and reception of the transmission signal and reception signal, a controller that controls up-and-down movement of the transducer array 30, and a controller that performs water pressure control of the preliminary tank 5 or temperature control of hot water.

The reception signal output from the transducer array 30 is stored in the storage unit 8 and is subjected to image processing computation by the image generating unit 7. The transmission image of the subject 1 which is generated by the image generating unit 7 is displayed on a monitor or the like provided in the input/output unit 9.

The controller 6, the image generating unit 7, and the storage unit 8 can be accommodated in a space below the bed 2.

The controller 6 is configured to include a processor (for example, a central processing unit (CPU) or a graphics processing unit (GPU)) and a memory in which a program is stored in advance, and the processor reads and executes a program, and thereby a function of the focal length adjusting unit 61 is executed by software. It is possible to realize a part or the entirety of the focal length adjusting unit 61 by software. For example, the focal length adjusting unit 61 may be configured using a custom IC such as an application specific integrated circuit (ASIC) or a programmable IC such as a field-programmable gate array (FPGA), or circuit design may be performed so as to realize such an operation.

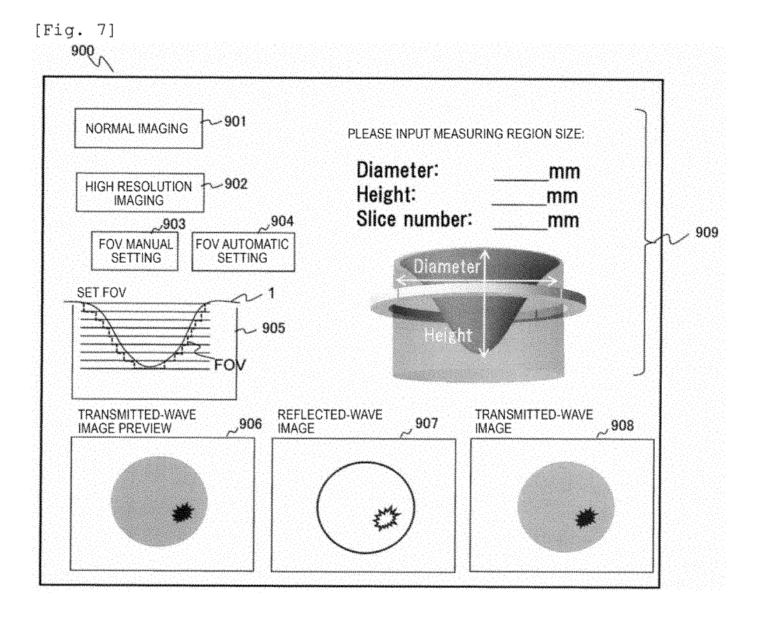

FIG. 6 is a sequence diagram illustrating transmission and reception of a signal of units. An overview of an operation of the ultrasonic CT apparatus of the embodiment is described with reference to FIG. 6. The controller 6 receives the reception signal of the reflected wave from the transducer 30 and data thereof is stored in the storage unit 8. The reflection image generating unit 7a of the image generating unit 7 receives data of the reception signal of the reflected wave from the storage unit 8, generates a reflection image, outputs the image to the focal length adjusting unit 61, and displays the image on the monitor of the input/output unit 9. The focal length adjusting unit 61 receives the reflection image from the reflection image generating unit 7a and sets transmission/reception conditions of the transmission, such as the length between the focal position 3c and the center 100a of the imaging space 100. The controller 6 receives the transmission/reception conditions of the transmission, causes the transducer array 30 to transmit the ultrasonic wave in the set transmission/reception conditions, and receives the transmission. In this manner, the ultrasonic wave of the fan beam having high intensity is transmitted from the plurality of transducers 3a and the transmission is received by the plurality of transducers 3c. The controller receives the reception signal from the transducer array and the data thereof is stored in the storage unit 8. The transmission image generating unit 7b receives items of reception signal data of the transmission from the storage unit 8 in the transmission/reception conditions such as the focal length from the focal length adjusting unit 61, reconstructs a transmission image, and displays the image on the monitor of the input/output unit 9. Since the transmission image is reconstructed from the reception signal data obtained from the fan beam having high intensity, a high-definition image is generated due to an effect of magnified projection and an effect of an increase in intensity of the reception signal.

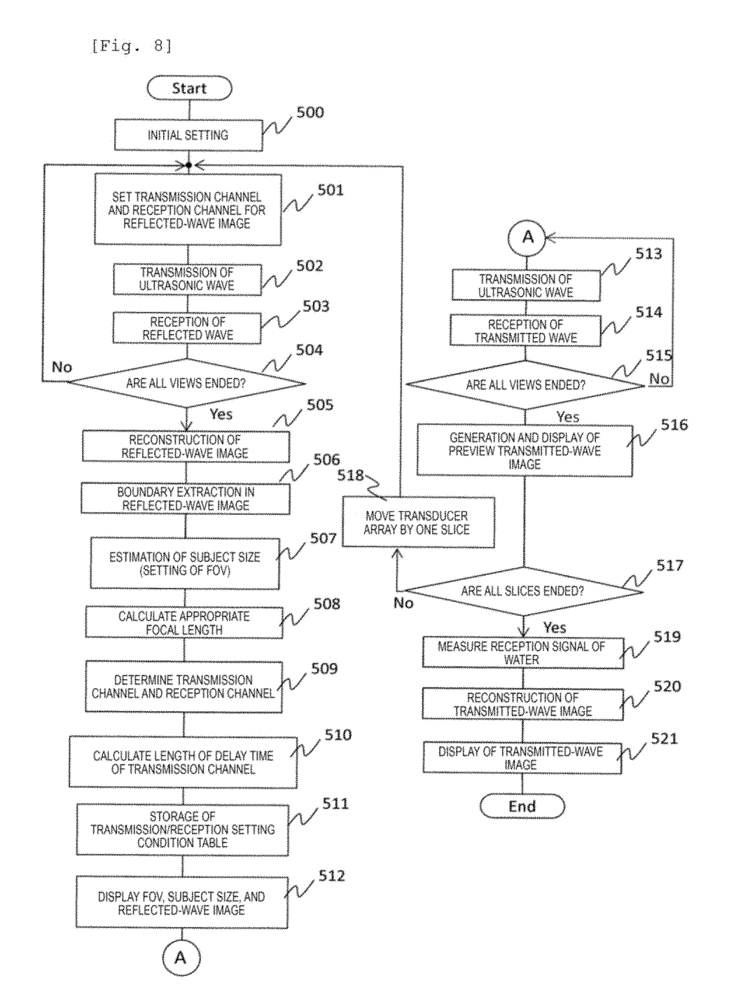

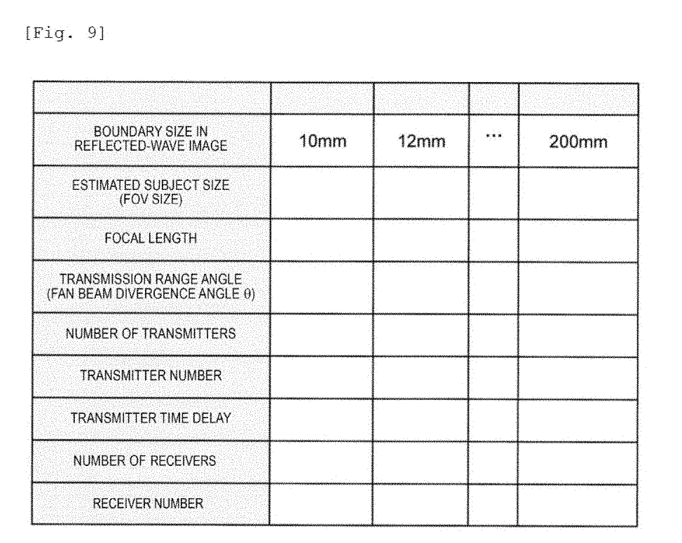

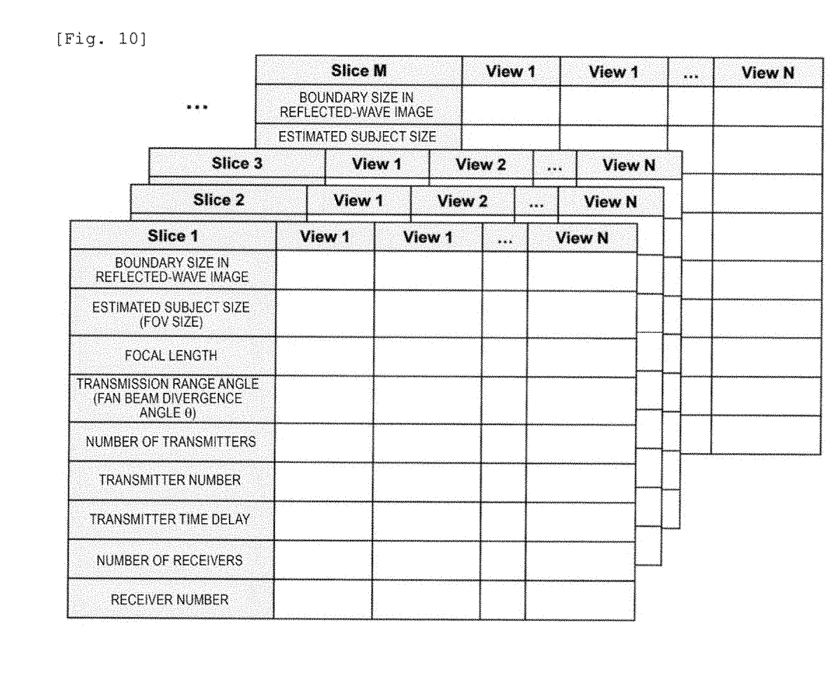

Hereinafter, the operation of the ultrasonic CT apparatus of the embodiment will be further described in detail. FIG. 7 is an example of a screen for receiving an operation by a user, and FIG. 8 is a flowchart illustrating an operation of the controller 6. FIG. 9 is a focal length reference table that is stored in a storage unit 8 in advance, and FIG. 10 is a transmission/reception setting condition table that is stored in the storage unit 8 in advance.

When the electric power is turned on, the controller 6 reads the temperature of water in the preliminary tank 5 from a thermometer, performs heating such that the temperature of the water is increased to a predetermined temperature (about the body temperature), causes a deaeration device to perform deaeration, and then a pump is driven so as to move the water to the water tub 4. In this manner, the water tub 4 is filled with deaerated water of which the temperature is adjusted to a predetermined temperature. In a state in which the subject 1 is not inserted, the controller 6 transmits and receives the ultrasonic waves in predetermined conditions, and the subject 1 acquires reception data of the deaerated water in the water tub.

In other words, the controller 6 displays a screen for an operation and display, such as a screen 900 in FIG. 7, on the monitor of the input/output unit 9. The screen 900 has buttons 901 and 902 for receiving which imaging of normal imaging or high-resolution imaging is performed by a user including the subject and an operator and buttons 903 and 904 for selecting, by the user, whether the setting of the FOV is manually or automatically performed in a case of the high-resolution imaging. In addition, the screen 900 has a region 905 for displaying the set FOV, a region 906 for displaying a preview of a rough transmission image, a region 907 for displaying the reflection image, a region 908 for displaying the transmission image, and a region 909 for inputting, by the user, a size of a measurement region in a case where the FOV manual setting 903 is selected by the user.

In a case where the user presses the button 901 or 902 for normal imaging or high-resolution image of the input/output unit 9, the controller 6 receives this and displays, on the monitor of the input/output unit 9, an indication for prompting the subject to change to an examination cloth and a screen for input the name or ID and a medical interview sheet of the subject on the monitor of the input/output unit 9. In this manner, when the subject 1 or the user inputs the name or the medial interview sheet on an input screen via the input/output unit 9, the controller 6 receives this and displays, on the monitor of the input/output unit 9, an indication for prompting the subject 1 to lie prone on the bed 2 and to input one breast on one side into the water tub 4. When the controller 6 confirms, through an operation of the input/output unit 9 by the subject 1, that the breast of the subject 1 is inserted into the water tub 4, the controller 6 determines whether the user presses the button 901 for the normal imaging or the button 902 for the high-resolution imaging, and the controller images the transmission image and the reflection image in a case where the button 901 for the normal imaging is pressed. On the other hand, in a case where the user presses the button 902 for the high-resolution imaging and the user presses the button 904 for the FOV automatic setting, steps from Step 501 are performed (Step 500).

In Steps 501 to 503, the controller 6 transmits and receives the ultrasonic waves to and from the transducer array 30 as will be described below. Here, the piezoelectric elements (ultrasonic elements 13) having 2,048 channels at a pitch of 0.5 mm are arranged in a ring shape, and the transducer array 30 having a diameter of 326 mm is configured. The water tub 4 of the piezoelectric element is 1 mm in thickness in the axial direction. The center frequency of the ultrasonic waves for irradiation from the transducer array 30 is 1.5 MHz (a wavelength of about 1 mm of the ultrasonic wave in water). First, the controller 6 sets a transmission channel and a reception channel for the reflection image and transmits and receives the ultrasonic waves. Specifically, the controller 6 sets, as the transmission channel and the reception channels (Step 501), the transducer of 512 channels continued in the transducer array 30, generates transmission signals obtained through delays by predetermined lengths of delay, and supplies the signal the transducers of transmission channels (Step 502). In this manner, irradiation is performed with phased plane-wave ultrasonic waves (parallel beams) from the transducer of 512 channels. The transducer of the same 512 channels as the transmission channels receives the reflected waves reflected from an imaging site (breast) of the subject 1 (Step 503). Then, it is possible to secure a diameter of 230 mm of a field of view (FOV). Steps 501 to 504 are repeatedly performed for the predetermined all views (Step 504). Specifically, while the controller 6 causes the transducer of 512 channels, which perform the irradiation with the ultrasonic waves on the transducer array 30 by shifting four channels at time, a transmitting operation and a receiving operation are repeatedly performed, and thereby signals of the reflected waves in a range of 360 degrees of the water tub 4 are obtained in 512 views shifted by 0.7 degrees in the slices (sections) of the depth of the water tub 4. The obtained reception signals of the reflected waves are stored in the storage unit 8.

The reflection image generating unit 7a of the image generating unit 7 reads received data of the reflected wave from the storage unit 8, a period of time (timing) from the irradiation with the ultrasonic wave from the transducer 3 to coming back of the ultrasonic wave to the transducer 3 from the subject 1 is obtained by dividing a sum of a length from the transmitted transducer 3 performing transmission to a pixel of interest (a point in the subject 1) and a length from the pixel of interest to the transducer 3 performing the receiving by a sound speed of the ultrasonic wave (for example, a sound speed of water). The reflection image generating unit 7a adds signals received by the ultrasonic elements at a timing when the reflected waves reflected from the pixel of interest in the subject 1 reach the ultrasonic elements performing the reception, and the signal intensity obtained after the addition is a value of the image. This method is referred to as a delay and sum (DAS). This operation is performed for all pixels in the field of view, and thereby the controller 61 reconstructs the reflection images for views. The reflection image generating unit 7a adds the reflection images obtained by the views, and thereby the reflection images of any slice of the subject 1 is generated (Step 505). The reflection image generating unit 7a displays the generated reflection image on the monitor of the input/output unit 9 and the image is stored in the storage unit 8.

In other words, the focal length adjusting unit 61 of the controller 6 performs image processing on the reflection image, obtains a boundary in the image, extracts the boundary corresponding to an outline of the subject 1, and obtains the size for each view (Step 506).

Here, the storage unit 8 stores a focal length reference table as illustrated in FIG. 9 which is obtained in advance. The table shows a corresponding relationship between a plurality of types of sizes of outlines of the subjects 1 obtained from the boundary of the reflection image, estimated sizes (FOVs) of the subject 1 obtained by anticipating an error due to refraction or the like of the ultrasonic wave with respect to the types of sizes, divergence angles θ corresponding to the FOVs, lengths (hereinafter, referred to as the focal length) between the focal position 3c and the center 100a of the imaging space for performing the irradiation with the ultrasonic waves of fan beams having the divergence angles θ, the number of transducers 3a that transmit the fan beams having the divergence angles θ, positions (numbers) of the transducers 3a for each view, delay times of the transmission signals which are supplied by the transducers 3a for superimposing the ultrasonic waves transmitted from the plurality of transducers 3a and generating the fan beams having the divergence angle θ, the number of transducers 3b that are positioned in a range of reach of the fan beams transmitted through the subject 1 and receive the transmissions, and positions (numbers) of the transducers 3b for each view.

In addition, as illustrated in FIG. 10, the storage unit 8 also stores a transmission/reception setting condition table for storing conditions of transmission and reception determined by the controller 6 for each view and each slice.

The focal length adjusting unit 61, with reference to the focal length reference table in FIG. 9 in the storage unit 8, obtains a focal length corresponding to the maximum value of the size of the boundary of the reflection image for each view obtained in Step 506, the divergence angle θ of the fan beam obtained in a case of the focal length, numbers of the plurality of transducers 3a which are transmission channels for transmitting the fan beams having the divergence angle θ for each view, numbers of the plurality of transducers 3b which are reception channels, and a length of delay time of the transducers 3a of transmission channels, selects the items as conditions for transmission and reception, and stores the items in the transmission/reception setting condition table illustrated in FIG. 10 (Steps 507 to 510). In addition, in order to perform a display for a user in accordance with the conditions for transmission and reception, an estimated size of the subject corresponding to the size of the boundary in the reflection image obtained in Step 506, the number of transducers 3a of transmission channels, and the number of transducers 3b of reception channels are also obtained from the table in FIG. 9 and are stored in the transmission/reception setting condition table illustrated in FIG. 10.

The focal length adjusting unit 61 displays, as an image at a slice position corresponding to the region 905 of the screen in FIG. 7, the FOV of the transmission/reception setting condition table stored in Step 511 and the boundary sizes of the reflection image (a size of the subject) and displays the reflection image generated in Step 505 in the region 907 (Step 512). The display method is not limited to an example of the screen in FIG. 7, and a transmission/reception setting condition table in FIG. 10 as it is may be displayed on the screen 900.

In other words, the controller 6 reads the number of any transducer 3a which is the transmission channel of the views and the delay time of the transmission signal for each transducer 3a from the transmission/reception setting condition table in storage unit 8, generates a transmission signal for each transducer 3a, outputs the signal to the transducers 3a obtained by delaying the signal by the set delay time, and transmits the ultrasonic waves (Step 513). In this manner, since the ultrasonic waves delayed by the predetermined length of delay are transmitted from the set plurality of transducers 3a, wave surfaces thereof are superimposed, and thereby the fan beams having the divergence angle θ are transmitted toward the subject 1. The transmission transmitted through the subject 1 is received by the transducer array. The controller 6 reads numbers of the transducers 3b which are reception channels from the transmission/reception setting condition table in FIG. 10, receives reception signal from the transducers 3b, and stores the signals in the storage unit 8 (Step 514). The controller 6 performs Steps 513 and 514 for all of the views (Step 515). In this manner, the fan beams having the same divergence angle θ are transmitted to the subject 1 for all of the views of a slice and acquires a reception signal of the transmission.

When the reception signals of the transmissions for all of the views are stored in the storage unit 8, the transmission image generating unit 7b of the image generating unit 7 generates and displays a preview of a rough transmission image obtained from the received reception signal in real time (Step 516). Specifically, the transmission image generating unit 7b reads the reception signal of the transmission from the storage unit 8, reads the conditions of the transmission/reception setting condition table, performs Hilbert transform in a time direction with respect to the reception signal, and obtains the maximum amplitude of the transmission and the timing thereof. The transmission image generating unit 7b calculates both of a difference in receiving times before and after the insertion of the subject 1 and a length in logarithm of the maximum amplitude for each view and each reception channel. At this time, as the logarithm of the receiving time and the maximum amplitude before the insertion of the subject 1, a value obtained from the reception signal obtained by transmitting and receiving the ultrasonic waves in the deaerated water in the water tub 4 before Step 500 is used. The transmission image generating unit 7b generates both of a sinogram of gathering of data of differences in receiving times and a sinogram of gathering of data of differences in the maximum amplitudes. The transmission image generating unit 7b performs a filtered back projection (FBP) or the like, which is widely used in the field of X-ray CT, on both of the sinogram of the differences in the receiving times and the sinogram of the differences in the maximum amplitudes, and thereby a tomographic image is reconstructed. A distribution image (tomographic image) of differences in “delay (slowness)” of the ultrasonic waves before and after the insertion of the subject 1 is obtained from the sinogram of the difference in the reception times. The “delay” is a reciprocal of the sound speed. A distribution image (tomographic image) of differences in attenuation rates before and after the insertion of the subject 1 is obtained from the sinogram of the differences in logarithm of the maximum amplitude. The transmission image generating unit 7b uses a predetermined value (estimated value) as the sound speed or the attenuation rate of water to generate images of a sound speed distribution and an attenuation rate distribution of the subject 1 from a distribution image of differences in the “delay (slowness)” and a distribution image of differences in the attenuation rate. The transmission image generating unit 7b displays the generated sound speed distribution image and attenuation distribution image in the region 906 of the screen 900 in FIG. 7 and stores the images in the storage unit 8.

In this manner, it is possible to obtain the reception signal by transmitting the fan beam having high intensity and the divergence angle θ depending on the size of the subject 1 of the reflection image and receiving the transmission for each slice, and it is possible to display previews of the transmission image and the reflection image. While the controller 6 displaces the transducer array 30 by one slice (for example, 0.5 mm) in the axial direction of the water tub 4, imaging in Steps 501 to 516 is repeated for all of the slices (for example, a total of 20 mm) (Steps 517 and 518). In this manner, when data of the reception signals by all of the slices (for example, 40 slices) of the subject is stored in the storage unit 8, the controller 6 moves the transducer array 30 to a predetermined depth close to the bottom of the water tub 4 below the subject 1, transmits the fan beams in the same transmission/reception conditions (conditions in the transmission/reception setting condition table in FIG. 10) for all of the slices, receives the transmissions, and then acquires the reception signal data of the deaerated water for the slices (Step 519). The transmission image generating unit 7b reads the data of the reception signal of the transmissions of all of the slices from the storage unit 8, reconstructs a transmission image (for example, a three-dimensional image) of the subject 1 with higher resolution than that in Step 516 by using the same method as in Step 516, and stores the image in the storage unit 8 (Step 520). However, in Step 519, as the logarithm of the receiving time and the maximum amplitude in a state in which the subject 1 is not inserted, a value obtained from the reception signal obtained in Step 519 is used. The transmission image generating unit 7b displays the transmission image of the subject 1, which is reconstructed in Step 521, in the region 908 of the screen 900.

The ultrasonic CT apparatus performs imaging by following the flow in FIG. 8, and thereby it is possible to transmit, from the plurality of transducers 3a, the fan beams having high intensity at the divergence angle θ depending on the size of the subject obtained from the reflection image. Therefore, it is possible to obtain a high definition transmission image.

In addition, the user can check a positional relationship between the set outline and FOV of the subject, by the display in the region 905 in FIG. 7, and it is advantageous that the preview of the transmission image in the region 906 and the reflection image in the region 907 is viewed and checked whenever imaging is performed.

In the flow in FIG. 8, since an operation of imaging the reflection image and the transmission image is performed in order for each slice, it is possible to continually image reflection images and transmission images for the same slice without a positional shift. In addition, since continual imaging of the reflection image and the transmission image is performed, it is advantageous that body movement of the subject or a change in water temperature is unlikely to influence both imaging operations.

In the flow in FIG. 8 of Embodiment 1, in Steps 501 to 505, the reflection image is reconstructed, the boundary is extracted from the reflection image in Step 506, and thereby the size of the subject 1 is detected. However, without Steps 501 to 505, the boundary may be extracted in Step 506 from a preview of the transmission image obtained in Step 516, the FOV may be set in steps after Step 507 based on the boundary, and a transmission image of the following slice may be imaged. Since the outline of the subject of the adjacent slices is considered to be continued, it is possible to set a substantially appropriate FOV even using a preview of the transmission image of the previous slice as described above. Accordingly, since it is possible to omit an imaging step of the reflection image, it is possible to image a high definition transmission image at a high speed.

### Embodiment 2

In the flow of Embodiment 1, a configuration in which the reflection image and the transmission image are imaged in order for each slice is described; however, the present invention is not limited thereto. The apparatus may have a configuration in which, first, the reflection images are imaged for all of the slices, the transmission/reception setting condition table in FIG. 10 is obtained by setting the FOV and the focal length, and then, in accordance with the conditions, the transmission images are imaged in order for all of the slices. In this case, setting results of the FOV or the focal length from the reflection image by the controller 6 for all of the slices are checked by the user through the display in the region 905 of the screen 900, and then it is possible to perform the imaging operation of the transmission image. Therefore, the apparatus may have a configuration in which, in a case where the user who views the setting results of the FOV or the focal length with the display in the region 905 wants to perform correction before imaging the transmission image, the controller 6 receives the correction from the user via the input/output unit 9. In this case, the controller 6 may correct a numerical value in the transmission/reception setting condition table in FIG. 10 into a numerical value received from the user via the input/output unit 9.

In addition, as another example, the controller 6 may first image the reflection images for all of the slices, set the FOV and the focal length of a slice having the largest diameter of the subject 1, set the FOV and the focal length, and then, image the transmission images for all of the slices. The controller 6 may be configured to display the obtained transmission image on the monitor, to receive a region of interest by the user, or to automatically set the region of interest through image processing, to perform processes in steps after Step 506 in FIG. 8 for only slices containing the region of interest, to set the optimal FOV and focal length, and to image the transmission image.

According to the example, an effect of shortening time taken for the processes of setting the FOV and the focal length can be obtained, compared to a configuration in which the optimal FOV and focal length are set for each slice.

### Embodiment 3

Embodiment 1 describes a case where the user presses the button 902 of the high-resolution imaging on the display screen in FIG. 7, and the user presses the button 904 of the FOV automatic setting; however, in Embodiment 3, a case where the user presses a button of the FOV manual setting 903 is described.

In a case where the user presses the button of the FOV manual setting 903, the focal length adjusting unit 61 of the ultrasonic CT apparatus displays the reflection image reconstructed in Step 505 in the region 907 of the screen 900 without performing the setting operation of boundary extraction from the reflection image and FOV based on the boundary extraction in Steps 506 and 507 in the flow in FIG. 8 of Embodiment 1. An input of the FOV size is received in the region 909 on the screen by the user. For example, a line surrounding a region for which the user wants to set FOV is manually drawn by using the input/output unit 9, or the like, and the focal length adjusting unit 61 receives the input of the FOV size by detecting the position and size of the region surrounded by the line. In this manner, it is possible to set a desired FOV size by a user.

The focal length adjusting unit 61 performs the processes in steps after Step 508 for the received FOV size. In this manner, it is possible to set appropriate focal length or the like for the FOV set by the user.

### Embodiment 4

In Embodiment 3, an example in which the user manually sets the FOV for each slice is described; however, in the embodiment, the focal length adjusting unit 61 may be configured to receive an input of a diameter of a proximal portion of the breast of the subject 1, a height from the proximal portion to a tip end, and the total number of slices set for the entire breast from the user in the region 909 of the screen 900 in FIG. 7. In this case, a plurality of shape models of the breast are stored in the storage unit 8.

The focal length adjusting unit 61 selects a breast model having the closest values to the input diameter of the proximal portion and the input height, obtains a diameter for each slice in a case where the model is sliced into the input total number of slices, and sets the FOV size. For the FOVs of the obtained slices, the processes after Step 508 in FIG. 8 may be performed.

In this manner, the user can relatively easily input the shape of the breast manually, and it is possible to set the FOV size.

In this configuration of the embodiment, there is no need to perform processes in Steps 501 to 507 of Embodiment 1; however, in a case where the user desires to display the reflection image, the reflection image may be generated by performing the processes in Steps 501 to 505.

### Embodiment 5

As Embodiment 5, an example, in which two types of ultrasonic beams (fan beams) having different lengths between the focal position 3c and the center 100a of the imaging space 100 are transmitted for the same slice, and a defect in the image is removed by receiving the reception signals of the respective transmissions is described. The image generating unit 7 generates the transmission image using the reception signal of the transmission which is obtained for each of the two types of ultrasonic beams.

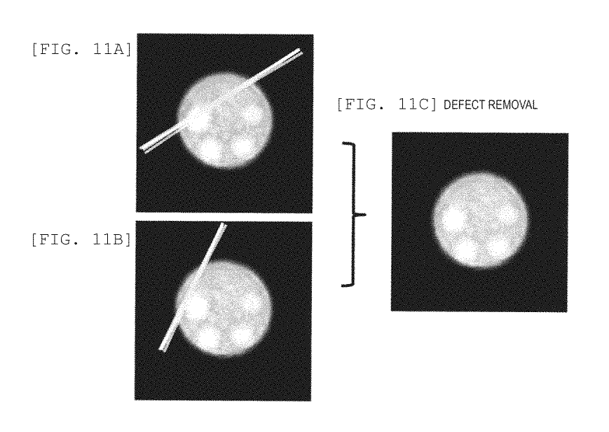

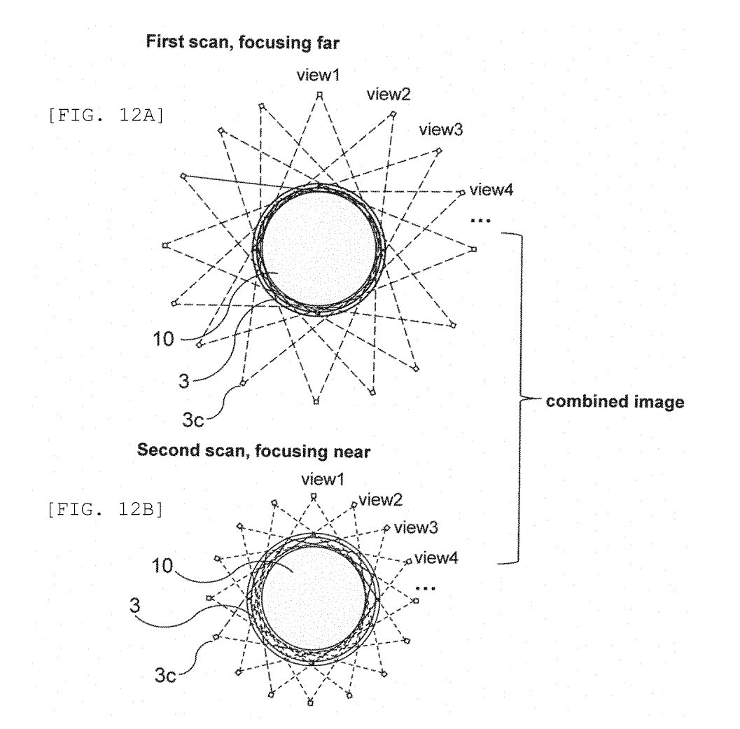

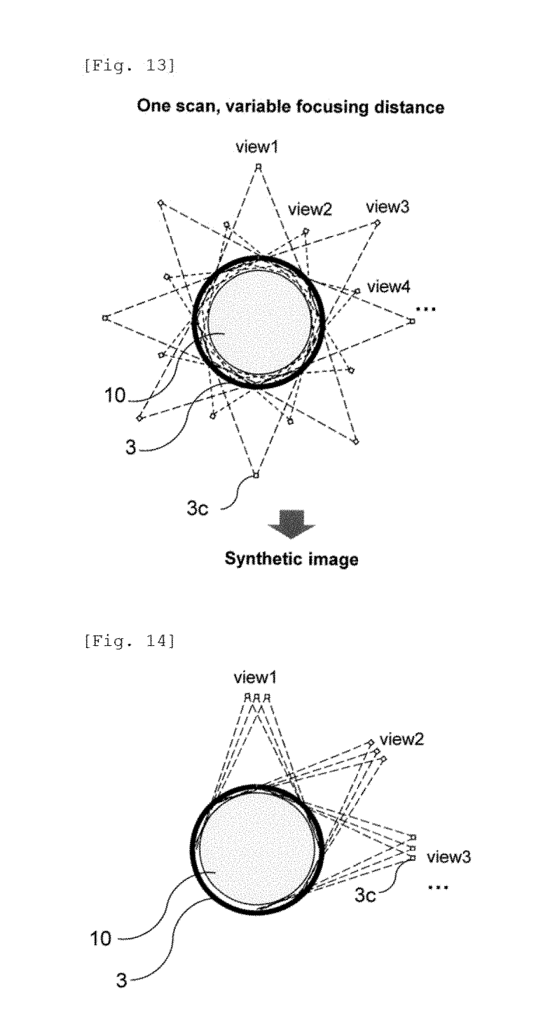

FIGS. 11(a) and 11(b) illustrate examples of images with a defect, and FIG. 11(c) illustrates an example of an image obtained by removing a defect. FIGS. 12(a) and 12 (b) are views illustrating a shape of a fan beam during imaging of a transmission image.

In general, in a case where one transducer 3 is out of order, or in a case where interference with a transmission path occurs due to a structure of the subject 1, a reconstructed transmission image is known to have a stripe-shape defect in FIGS. 11(a) and 11(b). In the embodiment, first, as illustrated in FIGS. 12(a) and 12 (b), the two types of fan beams having different lengths between the focal position 3c and the center 100a of the imaging space 100 are transmitted to the predetermined number of respective views having different incident angles for the same slice, and reception signals of the respective transmissions are received. The stripe-shaped defect is formed in the image (FIG. 11(a)) reconstructed from the reception signal obtained by using a fan beam having along focal length and the image (FIG. 11(b)) reconstructed from the reception signal obtained by using the fan beam having a short focal length, and angles of the defects are different from each other. This is because the divergence angles of the transmitted fan beams are different from each other. First, the image generating unit 7 detects the pixels in defect portions of the two generated images by image processing, and then the two images are combined with each other. In other words, any one of pixel values of pixels corresponding to the two images is selected, and two images are combined with each other. At this time, in a case where one pixel is a pixel of the defect in the other image, the pixel value of the pixel of the other image is selected. In this manner, since the defect positions of the two images are different from each other, it is possible to combine the images from which the defect is removed as illustrated in FIG. 11(c).

Since the FOV obtained in a case of using the fan beam having the short focal length is smaller than the FOV obtained in a case of using the fan beam having the long focal length, only an image of the reception signal obtained by using the fan beam having the long focal length is obtained in an outer circumferential portion of the FOV; however, the pixel values of the image obtained by using the fan beam having the long focal length may be selected during the combination. In this manner, it is possible to obtain an image for the large FOV.

Since, in an X-ray CT apparatus of the embodiment, it is possible to transmit a plurality of fan beams having different lengths between the focal position 3c and the center 100a of the imaging space 100, it is possible to remove the defect in the image.

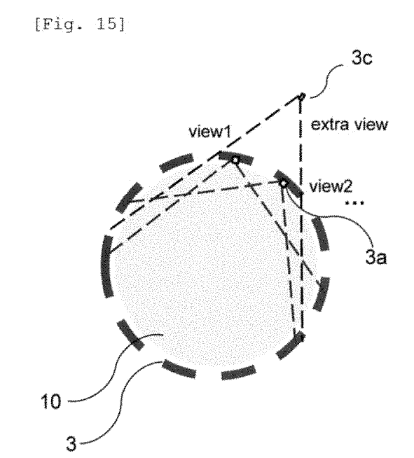

In the embodiment, an example in which two types of fan beams having different focal lengths are transmitted to the same views of the same slices and the reception signals are obtained is described; however, the embodiment is not limited to this configuration. As illustrated in FIG. 13, the irradiation with fan beams having different focal lengths alternately for each view for the same slices is performed. In this case, similar to FIGS. 11(a), 11(b), and 11(c), it is possible to reconstruct two images, and it is possible to combine the images from which the defect is removed.

In the embodiment, a configuration in which the two images are formed, and then combination of the images is performed while the defect is removed is described; however, a reception signal of a defective transducer is removed from the reception signals obtained by using the two types of respective fan beams, or the reception signal that passes through the defect portion is removed. Then, image reconstruction is performed, and thereby it is possible to generate an image obtained by removing the defect.

### Embodiment 6

The embodiment employs a configuration in which the ultrasonic waves that are transmitted from the plurality of transducers 3a are delayed by a predetermined length, then, wave surfaces are superimposed on each other, and the position of the focal position 3c is controlled to form the fan beams. Therefore, adjustment of the length of delay for each transducer 3a enables the focal positions 3c of the fan beams which are transmitted from the same plurality of transducers 3a to shift in an arrangement direction of the transducers 3a.

In Embodiment 6, a plurality of fan beams, of which the focal positions 3c slightly shift, are transmitted to the same views. In this manner, the X-ray CT apparatus realizes imaging of a flying focal spot which is well known in the X-ray CT apparatus.

FIG. 14 is a view illustrating a case where the X-ray CT apparatus images the flying focal spot. The focal position adjusting unit 61 of the controller 6 causes the transducer array 30 to transmit the ultrasonic beam the plurality of times for each of a plurality of views having different incident angles of the ultrasonic beam with respect to the imaging space 100 and to receive the reception signals. At this time, the focal length adjusting unit 61 sets the focal position 3c of the ultrasonic beam to a position that is farther away than the transducer array 30 when viewed from the center 100c of the imaging space, and moves the focal position of the ultrasonic beam, which is transmitted a plurality of times for each view, by a width which is equal to or less than a length between adjacent transducers 3a, in the arrangement direction of the transducers 3a.

The image generating unit 7 performs the image reconstruction and generates the transmission image using the reception signal of the transmission obtained for the ultrasonic beam which is transmitted a plurality of times for each view and an image reconstructing method of the known flying focal spot that is widely known in the X-ray CT apparatus.

In the ultrasonic CT apparatus of the embodiment, it is possible to generate the high definition transmission image by the effect of flying focal spot.

### Embodiment 7

As described in Embodiment 6, adjustment of the length of delay for each transducer 3a enables the focal positions of the fan beams which are transmitted from the transducers 3a to shift in the arrangement direction of the transducers 3a. In the embodiment, the number of views larger than the number of transducers 3 constituting the transducer array 30 is set and the view is irradiated with the fan beams.

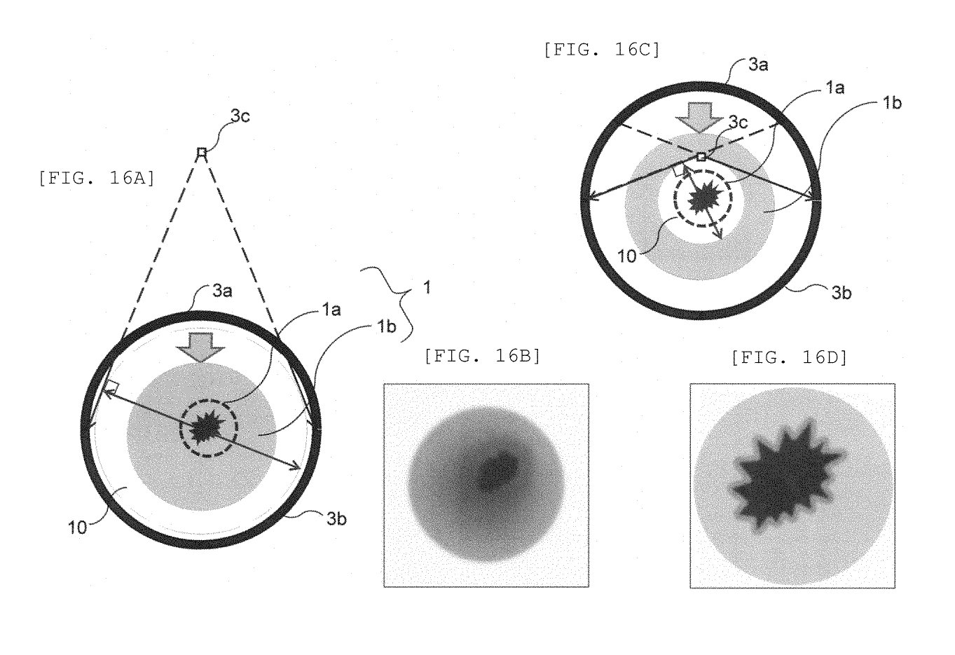

FIG. 15 illustrates a fan beam having the focal position 3c between the arranged transducers. In other words, the length of delay of the ultrasonic waves output from two or more adjacent transducers are adjusted using the transducers, and thereby it is possible to perform the irradiation with the fan beam having the focal position 3c between the transducer 3 and the transducer 3. In this manner, the controller 6 can set a plurality of views having different incident angles of the ultrasonic beams with respect to the imaging space 100 such that the number of views is larger than the number of transducers constituting the transducer array 30. The focal length adjusting unit 61 sets the focal position 3c of the ultrasonic beam to a position which is farther away than the transducer array 30 when viewed from the center 100a of the imaging space 100.

### Embodiment 8

In Embodiment 1, the fan beams, of which the focal positions are adjusted such that the entirety of the subject is placed within the divergence angle of the fan beam, are transmitted, and the transmission image of the entirety of the subject is reconstructed. However, in Embodiment 8, a region of interest (ROI) is set in the reconstructed transmission image, the fan beams, of which the focal lengths are adjusted such that only the ROI is placed within the divergence angle of the fan beam, are further transmitted, and then a transmission image of ROI is further reconstructed from the obtained reception signal. In this manner, since it is possible to irradiate only the ROI with the fan beams having high intensity, it is possible to obtain highly accurate image for the ROI.

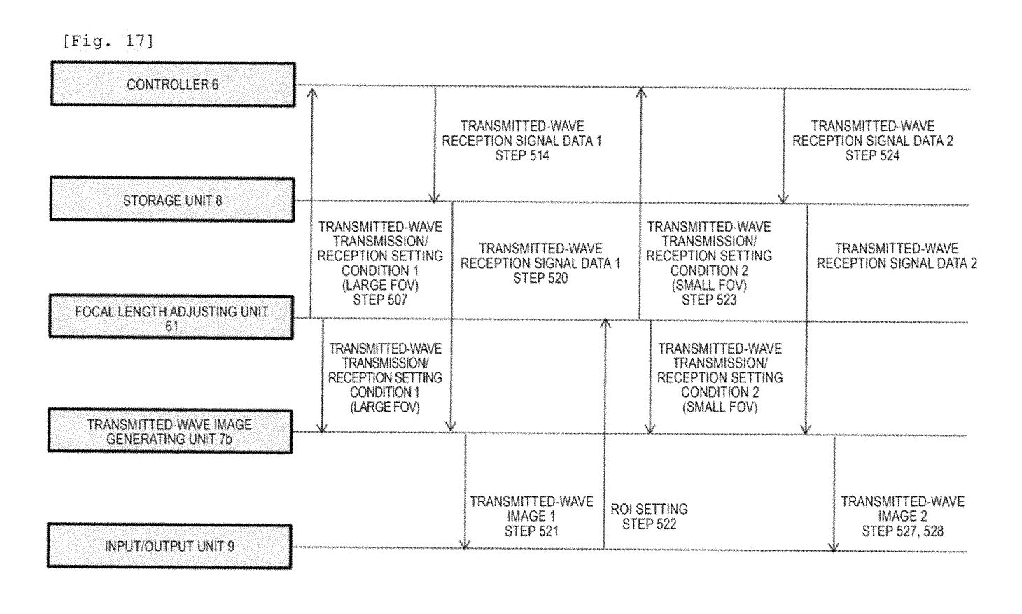

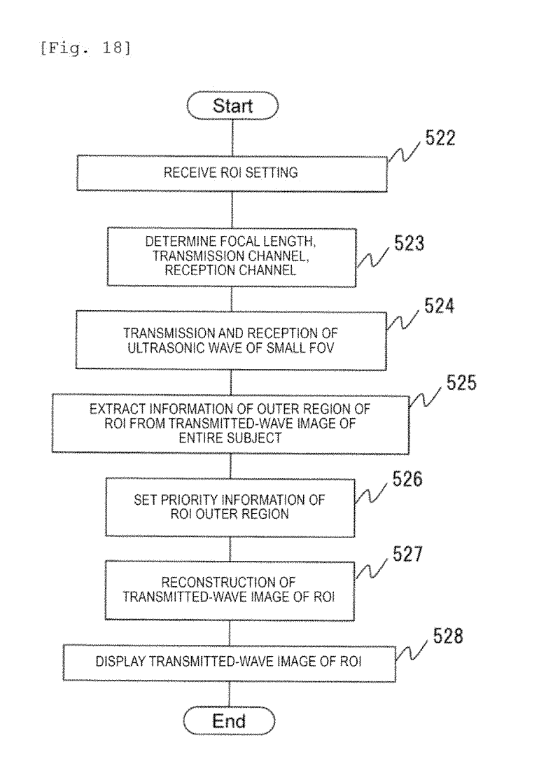

FIG. 16(a) illustrates irradiation with the fan beam of which the focal position 3c is adjusted depending on the FOV 10 that is large to the extent that all of the slices of the subject 1 are included, FIG. 16(b) illustrates an example of a transmission image of all of the obtained slices of the subject 1, FIG. 16(c) illustrates irradiation with the fan beam of which a focal position 3c is adjusted depending on the FOV that is small to the extent that only the ROI of the subject 1 is included, FIG. 16(d) illustrates an example of a transmission image of the ROI. FIG. 17 is a sequence diagram schematically illustrating the transmission and reception of the signal of units. FIG. 18 is a flowchart illustrating operations of units of Embodiment 8.

In the embodiment, similar to Embodiment 1, processes in Steps 500 to 521 in FIG. 8 are performed. In this manner, as illustrated in FIG. 17, the focal length adjusting unit 61 sets the FOV that is large to the extent that all of the slices of the subject 1 are included (Step 507) and sets the focal position 3c of the fan beam such that the FOV is included in the divergence angle (Step 508), and the controller 6 transmits the fan beam, of which the focal position 3c is adjusted, to the subject 1 (Step 513). As illustrated in FIG. 16(a), the ultrasonic waves of the transmitted fan beams have the divergence angle corresponding to the FOV in which all of the slices of the subject 1 are included. The controller 6 receives the reception signal of the transmission of the subject 1 and the signal is stored in the storage unit 8 (Step 514). The transmission image generating unit 7b reads the reception signal from the storage unit 8, reconstructs the transmission image of the subject 1, and displays the image on the monitor (Steps 520 and 521). The transmission image, which is displayed, of the subject 1 is an image of the entirety of the subject 1 as illustrated in the example in FIG. 16(b).

Subsequently, in Embodiment 8, the focal length adjusting unit 61, the controller 6, and the like operate as illustrated in the sequence diagram in FIG. 17 and the flow in FIG. 18. The focal length adjusting unit 61 receives, via the input/output unit 9, setting of a ROI 1a by a user who views the transmission image of the entirety of the subject 1 (Step 522). The focal length adjusting unit 61 sets the FOV that is small to the extent that only the ROI 1a is included, similar to the process in Step 508, with reference to the focal length reference table in FIG. 9 in the storage unit 8. In addition, similar to the processes in Steps 509 and 510, the focal length adjusting unit 61 obtains numbers of the plurality of transducers 3a which are the transmission channels, numbers of the plurality of transducers 3b which are the reception channels, and the length of time delay of the transducers 3a of the transmission channels, and sets the obtained results as the conditions for transmission and reception. The results are output to the controller 6 (Step 523). The controller 6 transmits the fan beam, of which the focal position 3c is adjusted, to the subject 1 in accordance with the received conditions of transmission and reception, obtains the reception signals of the transmissions, and the storage unit 8 stores the signal (Step 524). In this manner, as illustrated in FIG. 16(c), the fan beams are transmitted such that only the small FOV 10 is included, and the reception signal transmitted through the ROI is received.

The transmission image generating unit 7b is not capable of performing accurate image reconstruction because only the reception signal of the transmission of the ROI 1a obtained by the transmission of the ultrasonic beams of the small FOV from the storage unit 8 is not the reception signal data of the outer side of the small FOV. Information on an outer region 1b of the ROI 1a is extracted from the image (FIG. 16(b)) of the entirety of the subject 1 which is reconstructed in Step 520 (Step 528), and the transmission image of the ROI is reconstructed using the reception signal of the transmissions of the ROI 1a as prior information (Step 527). As a reconstruction method, for example, a method of generating an initial image required for the reconstruction of a ROI image from prior information on the entire image, a method of estimating a rough outline of the ROI portion from the entire image and setting the outline as the reconstruction conditions of the ROI image, a method of setting pixel values of the outer region 1b of the ROI 1a from prior information on the entire image and using the pixel values as the reconstruction conditions, a method of setting information on the outer region 1b of the ROI as a signal obtained by orthographic projection of the entire image, or the like is used. The transmission image generating unit 7b displays the reconstructed transmission image on the monitor of the input/output unit 9.

In this manner, as illustrated in FIG. 16(d), it is possible to obtain the enlarged high-definition image of the ROI 1a.

Embodiment 8 has a configuration in which the steps in FIG. 8 of Embodiment 1 are performed, and then the steps in FIG. 18, in which the irradiation is performed with the fan beams having the small FOV, are performed; however, the present invention is not limited thereto. After only specific slices of the subject are irradiated with the fan beams having the large FOV and the transmission image is obtained, the steps in FIG. 18 may be formed. In addition, the range of the irradiation with the fan beams having the large FOV may not be determined based on the reflection image, and the FOV having a predetermined size or the FOV having a size input by a user may be used.

### REFERENCE SIGNS LIST

1: subject
2: bed
3: transducer array
4: water tub
5: preliminary tank
6: controller
7: image generating unit
8: storage unit
9: input/output unit
7a: reflection image generating unit
7b: transmission image generating unit
61: focal length adjusting unit

1. An ultrasonic CT apparatus comprising:
a transducer array having an arrangement of a plurality of transducers that transmit an ultrasonic beam to a subject placed in a predetermined imaging space, receive a transmission of the ultrasonic beam transmitted through the subject, and output a reception signal;
an image generating unit that generates a transmission image based on the reception signal of the transducer array; and
a controller that controls the ultrasonic beam that is transmitted from the transducer array,
wherein the controller includes a focal length adjusting unit that adjusts a length between a focal position of the ultrasonic beam and the center of the imaging space to adjust a divergence angle of the ultrasonic beam.

2. The ultrasonic CT apparatus according to claim 1,
wherein the focal length adjusting unit is capable of setting a focal position of the ultrasonic beam to at least one of a position which is farther away than the transducer array and a position in the imaging space when viewed from the center of the imaging space.

3. The ultrasonic CT apparatus according to claim 1,
wherein the controller causes the plurality of transducers to transmit respective ultrasonic waves to the subject to superimpose the ultrasonic waves of the plurality of transducers and to form the ultrasonic beam, and the focal length adjusting unit adjusts a timing when the plurality of transducers transmit respective ultrasonic waves to adjust a length between the focal position of the ultrasonic beam and the center of the imaging space.

4. The ultrasonic CT apparatus according to claim 1,
wherein the image generating unit performs computation of image generation based on the divergence angle of the ultrasonic beam adjusted by the focal length adjusting unit when the transmission image is generated.

5. The ultrasonic CT apparatus according to claim 1,
wherein the focal length adjusting unit changes the length between the focal position and the center of the imaging space depending on a size of the subject such that the entirety of the subject is within the divergence angle of the ultrasonic beam.

6. The ultrasonic CT apparatus according to claim 5,
wherein the focal length adjusting unit obtains a size of the subject based on the image of the subject.

7. The ultrasonic CT apparatus according to claim 6,
wherein the transducer array further receives a reflected wave of the ultrasonic beam reflected from the subject,
wherein the image generating unit further generates a reflection image based on a reception signal of the reflected wave, and
wherein the focal length adjusting unit obtains a size of the subject based on the reflection image.

8. The ultrasonic CT apparatus according to claim 6, further comprising:
an input unit that receives a size of the subject by a user,
wherein the focal length adjusting unit changes the length between the focal position and the center of the imaging space depending on the size of the subject which is received via the input unit.

9. The ultrasonic CT apparatus according to claim 1,
wherein the image generating unit generates the transmission image for each of a plurality of slices set for the subject, and
wherein the focal length adjusting unit changes the length between the focal position and the center of the imaging space for each slice.

10. The ultrasonic CT apparatus according to claim 1,
wherein the controller transmits two types of ultrasonic beams having different lengths between the focal position and the center of the imaging space, and
wherein the image generating unit generates the transmission image using the reception signal of the transmission which is obtained for each of the two types of ultrasonic beams.

11. The ultrasonic CT apparatus according to claim 10,
wherein the controller transmits two types of ultrasonic beams for each of a plurality of views having different incident angles of the ultrasonic beam with respect to the imaging space, and
wherein the image generating unit generates transmission images for the respective two types of ultrasonic beams using the reception signal of the transmission obtained for each view to combine the two types of generated transmission images.

12. The ultrasonic CT apparatus according to claim 10,
wherein the controller alternately transmits the two types of ultrasonic beams for each of a plurality of views having different incident angles of the ultrasonic beam with respect to the imaging space.

13. The ultrasonic CT apparatus according to claim 1,
wherein the controller transmits an ultrasonic beam a plurality of times, for each of a plurality of views having different incident angles of the ultrasonic beam with respect to the imaging space,
wherein the focal length adjusting unit sets the focal position of the ultrasonic beam to a position that is farther away than the transducer array when viewed from the center of the imaging space, and moves, in an arrangement direction of the transducers, the focal position of the ultrasonic beam, which is transmitted a plurality of times for each view, by a width which is equal to or less than a length between adjacent transducers, and
wherein the image generating unit generates a transmission image using the reception signal of the transmission obtained for the ultrasonic beam which is transmitted a plurality of times for each view.

14. The ultrasonic CT apparatus according to claim 1,
wherein the controller sets a plurality of views having different incident angles of the ultrasonic beams with respect to the imaging space such that the number of views is larger than the number of transducers constituting the transducer array, and
wherein the focal length adjusting unit sets a focal position of the ultrasonic beam to a position which is farther away than the transducer array when viewed from the center of the imaging space to transmit the ultrasonic beam for each view.

15. The ultrasonic CT apparatus according to claim 1,
wherein the controller individually transmits a first ultrasonic beam, of which the focal position is adjusted such that the entirety of the subject is within the divergence angle of the ultrasonic beam, and a second ultrasonic beam, of which the focal position is adjusted such that only a region of interest inside the subject is within the divergence angle of the ultrasonic beam, and
wherein the image generating unit generates a transmission image of the region of interest using information on an outer region of the region of interest that is extracted from a reception signal of a transmission of the region of interest, which is obtained by the transmission of the second ultrasonic beam, and a reception signal of a transmission of the entirety of the subject, which is obtained by the transmission of the first ultrasonic beam.
